(19) United States
(12) Patent Application Publication
(10) Pub. No.: US 2023/0040247 A1
(43) Pub. Date: Feb. 9, 2023

(54) COMPOUNDS FOR TREATING NEURODEGENERATIVE DISEASES

(71) Applicant: Amabiotics, Paris (FR)

(72) Inventors: Nicole McKnight, New York, NY (US); Patricia Richard, new York, NY (US); Xavier Maniere, Montrouge (FR); Antoine Danchin, Paris (FR); Patrice Garnier, Venezia (IT)

(21) Appl. No.: 17/641,197

(22) PCT Filed: Sep. 12, 2020
(86) PCT No.: PCT/EP2020/075581

§ 371 Date: Mar. 8, 2022

(30) Foreign Application Priority Data
Sep. 12, 2019 (EP) 19197139.9

(51) Int. Cl. A61K 31/7068; A61K 45/06; A61P 25/28

(52) U.S. Cl. CPC A61K 31/7068; A61K 45/06; A61P 25/28

(57) ABSTRACT

The present invention relates to a compound of the following formula (I): or a pharmaceutically acceptable salt or hydrate thereof, for use in the prevention or treatment of a neurodegenerative disease selected from the group consisting of a-synucleinopathy, in particular Lewy body disease, Huntington's disease, frontotemporal neurocognitive disorder, and amyotrophic lateral sclerosis in an individual.

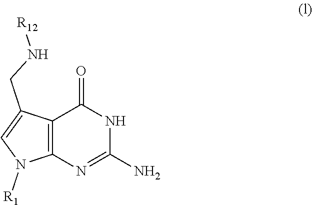

### FIELD OF THE INVENTION

The present invention relates to compounds, compositions and methods for treating neurodegenerative diseases, in particular alpha-synucleinopathy (a-synucleinopathy), such as Lewy body disease, Huntington's disease, frontotemporal neurocognitive disorder, and amyotrophic lateral sclerosis.

### BACKGROUND OF THE INVENTION

Progressive cognitive impairment is a core feature of a-synucleinopathies, such as Lewy body disease, of Huntington's disease, of frontotemporal neurocognitive disorder, and of amyotrophic lateral sclerosis (ALS). The impaired cognition represents a decline from a previously attained level of functioning and affects memory, thinking, reasoning and in some cases the motor system.

These disorders can be categorized and diagnosed as either major or mild, depending on the severity of an individual's symptoms. Major cognitive disorder is estimated to affect 1 to 2 percent of people by age 65 and as much as 30 percent of the population by age 85.

Alpha-synucleinopathies are characterised by the abnormal accumulation of a-synuclein aggregates in neurons, nerve fibres or glial cells. Lewy body disease is one of the main types of a-synucleinopathy. This disease include progressive cognitive impairment, complex visual hallucinations and concurrent symptoms of rapid eye movement (REM), sleep behavior disorder as well as hallucinations in other sensory modalities, depression, and delusions.

There is currently no treatment to cure Lewy body disease in itself. Indeed, the drugs used in this context, such as cholinesterase inhibitors (e.g. rivastigmine, donepezil and galantamine) or carbidopa-levodopa, only aim at alleviating the symptoms of the disease. In addition, the management can be complex because of the need to balance treatment of cognitive dysfunction, neuropsychiatric features, impairments related to the motor system, and other nonmotor symptoms. Besides, these compounds are not deprived of side effects.

Accordingly, there is still a need for alternative treatments of Lewy body disease, in particular by altering the pathological mechanisms of this condition in addition to treating the symptoms.

### SUMMARY OF THE INVENTION

The present invention arises from the recognition, by the present inventors, of the potential of queuine as a neuroprotective agent in the prevention, notably the prevention of symptoms, or treatment of neurodegenerative diseases, notably a-synucleinopathy, in particular Lewy body disease, Huntington's disease, frontotemporal neurocognitive disorder, and amyotrophic lateral sclerosis.

Thus, the present invention relates to queuine, a precursor of queuine, a derivative of queuine or a stereoisomer of queuine, an analogue of queuine, or a pharmaceutically acceptable salt or hydrate thereof, for use in the prevention or treatment of neurodegenerative diseases, in particular selected from the group consisting of a-synucleinopathy, in particular Lewy body disease, Huntington's disease, frontotemporal neurocognitive disorder, and amyotrophic lateral sclerosis, in an individual.

The present invention relates to a compound of the following formula (I):

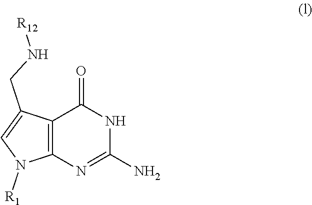

wherein:
R₁ represents —H or a ribosyl group of the following formula:

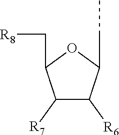

wherein:
   R₆ represents —H; —O—R₉ or —O—CO—R₉ wherein R₉ is H, an alkyl group having from 1 to 6 carbon atoms or an aryl group having from 3 to 12 carbon atoms;
   R₇ represents —H; —O—R₁₀ or —O—CO—R₁₀ wherein R₁₀ is H, an alkyl group having from 1 to 6 carbon atoms or an aryl group having from 3 to 12 carbon atoms; a deoxyribonucleic acid group; or a ribonucleic acid group;
   R₈ represents —H; —O—R₁₁ or —O—CO—R₁₁ wherein R₁₁ is H, an alkyl group having from 1 to 20 carbon atoms or an aryl group having from 3 to 20 carbon atoms; a phosphate group; a diphosphate group; a triphosphate group; a deoxyribonucleic acid group; or a ribonucleic acid group;
R₁₂ represents a saturated or unsaturated alkyl, cycloalkyl, heterocycloalkyl or ether group having from 1 to 20 carbon atoms, optionally substituted by at least one group selected from the group consisting of:
   an alkyl group having from 1 to 20 carbon atoms,
   an aryl or heteroaryl group having from 3 to 20 carbon atoms,
   a cycloalkyl or heterocycloalkyl group having from 3 to 20 carbon atoms,
   a hydroxyl group,
   a carbonyl or carboxyl group having from 1 to 20 carbon atoms,
   an epoxy group,
   an —O—R₄ group wherein R₄ is H, an alkyl group having from 1 to 6 carbon atoms, an aryl group having from 3 to 12 carbon atoms, a glycosyl group or an aminoacyl group,
   an —O—CO—R₅ group wherein R₅ is an alkyl group having from 1 to 6 carbon atoms, an aryl group having from 3 to 12 carbon atoms or a glycosyl group;
      or a pharmaceutically acceptable salt or hydrate thereof,
      for use in the prevention or treatment of a neurodegenerative disease, in particular selected from the group consisting of a-synucleinopathy, in particular Lewy body disease, Huntington's disease, frontotemporal neurocognitive disorder, and amyotrophic lateral sclerosis, in an individual.

The present invention also relates to a compound of formula (I) having the following formula (II):

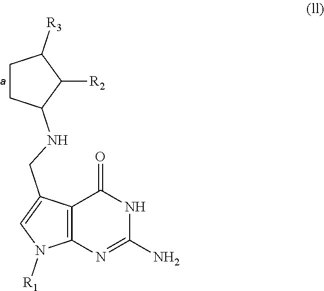

wherein:
a represents a double bond or an epoxy group, and
R₁ represents —H or a ribosyl group of the following formula:

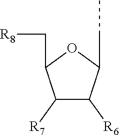

wherein:
   R₆ represents —H; —O—R₉ or —O—CO—R₉ wherein R₉ is H, an alkyl group having from 1 to 6 carbon atoms or an aryl group having from 3 to 12 carbon atoms;
   R₇ represents —H; —O—R₁₀ or —O—CO—R₁₀ wherein R₁₀ is H, an alkyl group having from 1 to 6 carbon atoms or an aryl group having from 3 to 12 carbon atoms; a deoxyribonucleic acid group; or a ribonucleic acid group;
   R₈ represents —H; —O—R₁₁ or —O—CO—R₁₁ wherein R₁₁ is H, an alkyl group having from 1 to 20 carbon atoms or an aryl group having from 3 to 20 carbon atoms; a phosphate group; a diphosphate group; a triphosphate group; a deoxyribonucleic acid group; or a ribonucleic acid group;
R₂ and R₃, which are identical or different, represent —O—R₄ wherein R₄ is H, an alkyl group having from 1 to 6 carbon atoms, an aryl group having from 3 to 12 carbon atoms, a glycosyl group or an aminoacyl group; or —O—CO—R₅ wherein R₅ is an alkyl group having from 1 to 6 carbon atoms, an aryl group having from 3 to 12 carbon atoms or a glycosyl group;
   or a pharmaceutically acceptable salt or hydrate thereof,
   for use in the prevention or treatment of neurodegenerative disease, in particular selected from the group consisting of a-synucleinopathy, in particular Lewy body disease, Huntington's disease, frontotemporal neurocognitive disorder, and amyotrophic lateral sclerosis, in an individual.

In an embodiment of the invention the compound of formula (I), in particular the compound of formula (II), or the pharmaceutically acceptable salt or hydrate thereof, for use as defined above, is in combination with at least one additional compound useful for the prevention or treatment of a neurodegenerative disease, in particular selected from the group consisting of a-synucleinopathy, in particular Lewy body disease, Huntington's disease, frontotemporal neurocognitive disorder, and amyotrophic lateral sclerosis.

The present invention also relates to a pharmaceutical composition comprising as active substance a compound of formula (I), in particular a compound of formula (II), or a pharmaceutically acceptable salt or hydrate thereof as defined above, optionally in association with at least one pharmaceutically acceptable excipient or vehicle, for use in the prevention or treatment of a neurodegenerative disease in particular selected from the group consisting of a-synucleinopathy, in particular Lewy body disease, Huntington's disease, frontotemporal neurocognitive disorder, and amyotrophic lateral sclerosis, in an individual.

In an embodiment of the present invention the above defined pharmaceutical composition further comprises at least one additional compound useful for the prevention or treatment of a neurodegenerative disease, in particular selected from the group consisting of a-synucleinopathy, in particular Lewy body disease, Huntington's disease, frontotemporal neurocognitive disorder, and amyotrophic lateral sclerosis.

The present invention also relates to a pharmaceutical composition comprising as active substance a compound of formula (I), in particular a compound of formula (II), or a pharmaceutically acceptable salt or hydrate thereof, as defined above, further comprising at least one additional compound useful for the prevention or treatment of a neurodegenerative disease, in particular selected from the group consisting of a-synucleinopathy, in particular Lewy body disease, Huntington's disease, frontotemporal neurocognitive disorder, and amyotrophic lateral sclerosis, optionally in association with at least one pharmaceutically acceptable excipient or vehicle.

The present invention also relates to products comprising:
a compound of formula (I), in particular a compound of formula (II), or a pharmaceutically acceptable salt or hydrate thereof, as defined above,
at least one additional compound useful for the prevention or treatment of a neurodegenerative disease, in particular selected from the group consisting of a-synucleinopathy, in particular Lewy body disease, Huntington's disease, frontotemporal neurocognitive disorder, and amyotrophic lateral sclerosis,
   as a combined preparation for simultaneous, separated or sequential use in the prevention or treatment of a neurodegenerative disease, in particular selected from the group consisting of a-synucleinopathy, in particular Lewy body disease, Huntington's disease, frontotemporal neurocognitive disorder, Lewy body disease and amyotrophic lateral sclerosis, in an individual.

The present invention also relates to a dietary supplement comprising a compound of formula (I), in particular a compound of formula (II), or a pharmaceutically acceptable salt or hydrate thereof, as defined above, for use for reducing the risk of a neurodegenerative disease, in particular selected from the group consisting of a-synucleinopathy, in particular Lewy body disease, Huntington's disease, frontotemporal neurocognitive disorder, and amyotrophic lateral sclerosis.

In an embodiment of the present invention, the dietary supplement as defined above, optionally comprises additional compounds, preferably selected from the group consisting of vitamins, minerals, fatty acids, amino acids and antioxidants.

The present invention also relates to a method for the prevention or treatment of a neurodegenerative disease, in particular selected from the group consisting of a-synucleinopathy, in particular Lewy body disease, Huntington's disease, frontotemporal neurocognitive disorder, and amyotrophic lateral sclerosis, in an individual, comprising administering to the individual an effective amount of a compound of formula (I), in particular of a compound of formula (II), or of a pharmaceutically acceptable salt or hydrate thereof, as defined above.

In an embodiment of the present invention, the method as defined above further comprises the administration of at least one compound useful for the prevention or treatment of a neurodegenerative disease, in particular selected from the group consisting of a-synucleinopathy, in particular Lewy body disease, Huntington's disease, frontotemporal neurocognitive disorder, and amyotrophic lateral sclerosis.

The present invention also relates to the use of a compound of formula (I), in particular a compound of formula (II), as defined above for the manufacture of a medicament intended for the prevention or treatment of a neurodegenerative disease, in particular selected from the group consisting of a-synucleinopathy, in particular Lewy body disease, Huntington's disease, frontotemporal neurocognitive disorder, and amyotrophic lateral sclerosis, in an individual.

In an embodiment of the present invention, the medicament as defined above further comprises at least one compound useful for the prevention or treatment of a neurodegenerative disease, in particular selected from the group consisting of a-synucleinopathy, in particular Lewy body disease, Huntington's disease, frontotemporal neurocognitive disorder, and amyotrophic lateral sclerosis.

### DETAILED DESCRIPTION OF THE INVENTION

### Compound of Formula (I)

Compounds of formula (I) as defined above can be readily chemically synthesized by one of skilled in the art, as is in particular described in Barnett & Grubb (2000), Tetrahedron 56: 9221-9225, Oxenford et al. (2004) Tetrahedron Letters 45:9053-9055, Brooks et al. (2010) Tetrahedron Letters 51: 4163-4165, Gerber et al. (2012) Org. Biomol. Chem. 10: 8660-8668, the thesis by Allen Brook entitled “Synthesis of Tritium Labeled Queuine, PreQ₁ and Related Azide Probes Toward Examining the Prevalence of Queuine” (2012, University of Michigan), Akimoto et al. (1986) J. Med. Chem., 29: 1749-1753, Kelly et al. (2016) Nucleic Acids Research, 1-11, and international application WO2016/050806, all of which are incorporated herein by reference.

Briefly, by way of example, queuine can be synthesised according to the following reaction scheme:

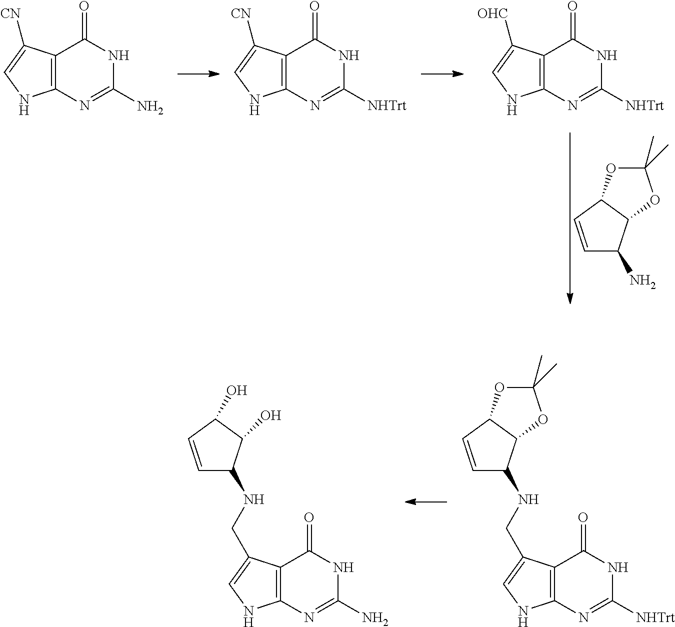

In additions, compounds of formula (I) as defined above may be extracted and optionally purified from natural sources such as microorganisms, in particular bacteria, or from plants, in particular from plants modulated with alpha-Proteobacteria such as bacteria of the Rhizobium, Mesorhizobium, and Sinorhizobium genii.

By way of example, queuosine can be obtained from tRNAs, in particular tRNA^{ASn}, tRNA^{Asp}, tRNA^{His} and tRNA^{Tyr}, prepared as follows:

Preparation of Total RNA Under Acidic Conditions
B. subtilis strains (or other relevant bacteria) are grown in ED liquid medium with appropriate supplements at 37° C. with constant aeration. Fresh overnight cultures are inoculated in 15 ml of ED medium to an optical density at 600 nm (OD600) of 0.1. Cells are grown at 37° C. to OD600 of 1 and chilled in equal volume of 60% methanol in 70 mM Hepes pH 7.5 at −80° C. All subsequent steps are carried out in the cold and the solution for prepared crude RNA are treated with diethyl pyrocarbonate and sterilized. Cells are pelleted at 4° C., washed in water and re-suspended in 0.5 ml of 10% glucose, 11 mM Tris, 10 mM EDTA. The suspensions are transferred to tubes containing 0.1 g glass beads acid-washed (sigma-Aldrich, G4649). Tubes are disposed into The CoolPrep Adapter of FastPrep®-24 Instrument (MP Biomedicals) containing 50 g of dry ice. Cells are broken after three cycles using following parameters: 6 meters per second during 45 s. After each cycle, suspensions are kept 1 min on ice. After centrifugation 2 min at 10,000 rpm, the supernatants are transferred to a fresh Eppendorf tube. 0.3 M sodium acetate pH 5.2 is added and total RNA is isolated under acidic conditions. One volume of acid phenol:chloroform with Isoamyl alcohol (125:24:1) pH 4.5 (Amresco, AM9720) is added. The sample are mixed by vortexing 10 s and incubated for 3 min in a 65° C. water-bath. The phases are separated by spinning 5 min at 14,000 rpm, then the aqueous phase is re-extracted once with the same hot acid phenol procedure. The aqueous phase is transferred to a new tube and supplemented by one volume of cold acid phenol. After centrifugation 5 min at 14,000 rpm, RNA are precipitated with 2.5 volumes of absolute ethanol 1 h at −80° C. The RNA is pelleted at 14,000 rpm for 15 min at 4° C. and washed with 70% ethanol. The RNA pellet is dissolved in 10 mM Tris, 1 mM EDTA pH 7.5.

Enrichment of tRNA
The total RNA preparation is then mixed with one volume of lithium chloride 8 M pH 4.5 and sodium acetate pH 5.2 at 0.01 mM final concentration. This RNA solution is incubated 2 h at −80° C. After centrifugation at 14,000 rpm for 15 min at 4° C., the tRNA remained in supernatant. To remove salt contamination, tRNA is precipitated 1 h at −80° C. by addition of 0.3 M sodium acetate pH 5.2 and 2.5 volumes of absolute ethanol. Then, tRNA is pelleted by centrifugation at 14,000 rpm for 15 min at 4° C. and washed with 70% ethanol. The tRNA pellet is dissolved in 10 mM Tris, 1 mM EDTA pH 7.5.

The stereoisomer of queuine according to the invention can be of any type. Preferably, the stereoisomer of queuine is ent-queuine.

The pharmaceutical acceptable salt or hydrate according to the invention can be of any type. However, it is preferred that the pharmaceutical acceptable salt according to the invention is a hydrochloride salt.

Preferably, the glycosyl group according to the invention is selected from the group consisting of a mannosyl group, a galactosyl group or a glutamyl group.

Preferably, the aminoacyl group is selected from alanine (ala, A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y) and valine (val, V).

In an embodiment of the invention, the substituents of formula (I), in particular of formula (II), according to the invention may be linked together.

In a preferred embodiment of the compound of formula (I) as defined above:
R₁ is H, and
R₁₂ represents a saturated or unsaturated alkyl, cycloalkyl, heterocycloalkyl or ether group having from 1 to 20 carbon atoms, optionally substituted by at least one group selected from the group consisting of:
   an alkyl group having from 1 to 20 carbon atoms,
   an aryl or heteroaryl group having from 3 to 20 carbon atoms,
   a cycloalkyl or heterocycloalkyl group having from 3 to 20 carbon atoms,
   a hydroxyl group,
   a carbonyl or carboxyl group having from 1 to 20 carbon atoms,
   an epoxy group,
   an —O—R₄ group wherein R₄ is H, an alkyl group having from 1 to 6 carbon atoms, an aryl group having from 3 to 12 carbon atoms, a glycosyl group or an aminoacyl group,
   an —O—CO—R₅ group wherein R₅ is an alkyl group having from 1 to 6 carbon atoms, an aryl group having from 3 to 12 carbon atoms or a glycosyl group.

In another preferred embodiment of the compound of formula (I) as defined above:
R₁₂ represent a group of the following formula:

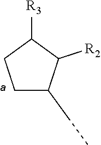

wherein:
a represents a double bond or an epoxy group, and
R₂ and R₃, which are identical or different, represent —O—R₄ wherein R₄ is H, an alkyl group having from 1 to 6 carbon atoms, an aryl group having from 3 to 12 carbon atoms, a glycosyl group or an aminoacyl group; or —O—CO—R₅ wherein R₅ is an alkyl group having from 1 to 6 carbon atoms, an aryl group having from 3 to 12 carbon atoms or a glycosyl group.

In another preferred embodiment of the compound of formula (I), as defined above:
R₁ is H, and
R₁₂ represents a saturated or unsaturated alkyl group having from 1 to 20 carbon atoms, optionally substituted by at least one of a hydroxyl group.

In another preferred embodiment of the compound of formula (I), in particular of the compound of formula (II), as defined above:
R₂ and R₃, which are identical or different, represent —OH, a —O-mannosyl group, a —O-galactosyl group or a —O-glutamyl group;
R₆ represents —OH;
R₇ and R₈, which are identical or different, represent —OH or a ribonucleic acid group.

Preferably, when R₇ and R₈ both represent a ribonucleic acid group, the compound of formula (I) according to the invention is included in a transfer RNA (tRNA) as a ribonucleoside of the tRNA. More preferably, the compound of formula (I) according to the invention is a ribonucleoside of the anticodon of the tRNA, most preferably the first nucleoside of the anticodon, i.e. the 5′ nucleoside of the anticodon or the nucleoside in the wobble position of the anticodon. Preferred tRNAs according to the invention are selected from the list consisting of tRNA^{Asn}, tRNA^{Asp}, tRNA^{His} and tRNA^{Tyr}.

Preferably, the compound of formula (I), in particular the compound of formula (II), as defined above is represented by the following formulae (III), (IV) or (V):

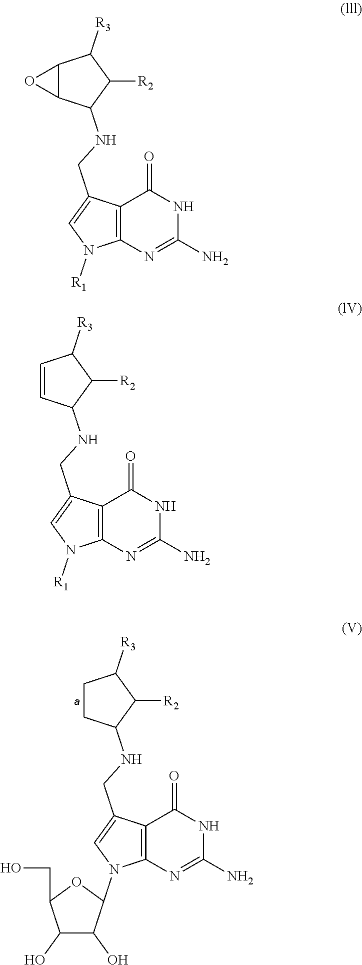

Preferably, when a compound of formula (I), in particular a compound of formulae (III)-(V) according to the invention is included in a tRNA^{Asp}, then R₃ is OH and R₂ is O-mannose.

Preferably also, when a compound of formula (I), in particular a compound of formulae (III)-(V) according to the invention is included in a tRNA^{Tyr}, then R₃ is OH and R₂ is O-galactose.

Preferably, the compound of formula (I), in particular the compound of formula (II) according to the invention is represented by the following formula (VI):

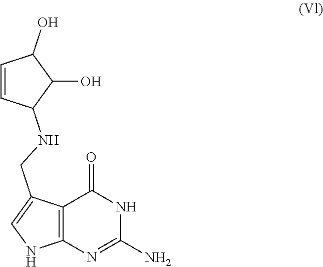 [SMILES: Nc1nc2[nH]cc(CNC3C=CC(O)C3O)c2c(=O)[nH]1]

As should be clear to one of skilled in the art, all the stereochemical configurations of the compounds according to the invention are intended to be covered by the formulae shown herein. In particular, as is intended herein, when the stereoconfiguration of a bond is not specified, the bond may represent any of an upward bond, a downward bond, and a mixture of the two, in particular a 1/1 mixture of the two.

Thus, the compound of formula (I) according to the invention also relates to the optically active forms of the compound of formula (V), such as the enantiomers represented by the following formulae (Va) and (Vb):

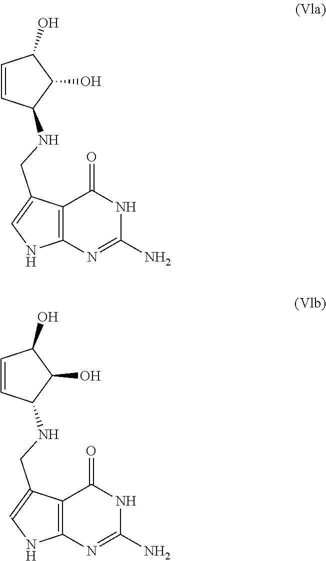 [SMILES: Nc1nc2[nH]cc(CN[C@H]3C=C[C@H](O)[C@@H]3O)c2c(=O)[nH]1 ; Nc1nc2[nH]cc(CN[C@@H]3C=C[C@@H](O)[C@H]3O)c2c(=O)[nH]1]

or their mixtures, in particular a racemic mixture thereof.

The compound of formula (VIa) is queuine. Queuine, is also known as 7-(3,4-trans-4,5-cis-dihydroxy-1-cyclopenten-3-ylaminomethyl)-7-deazaguanine. The compound of formula (VIb) is ent-queuine.

Preferably, the compound of formula (I), notably the compound of formula (II) according to the invention is represented by the following formulae (VII), (VIIa), (VIIb), (VIII), (VIIa), (VIIIb), (IX), (IXa) (IXb), (X)

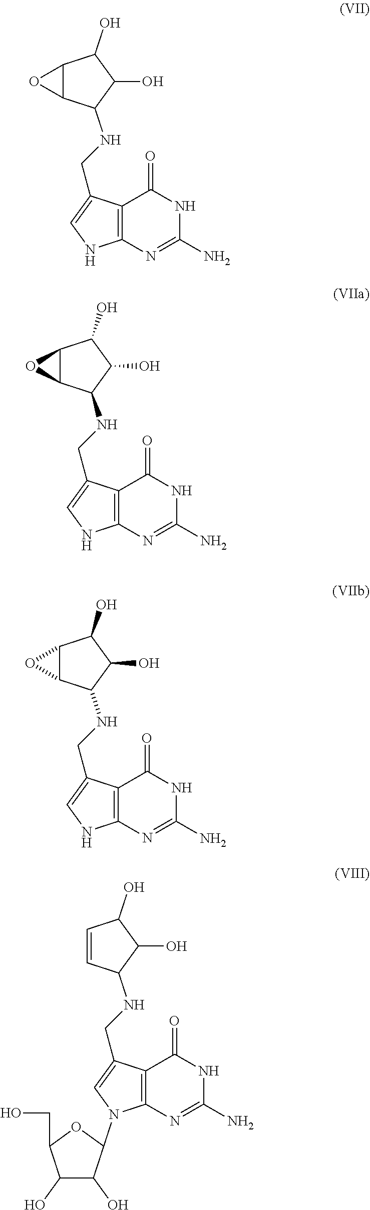

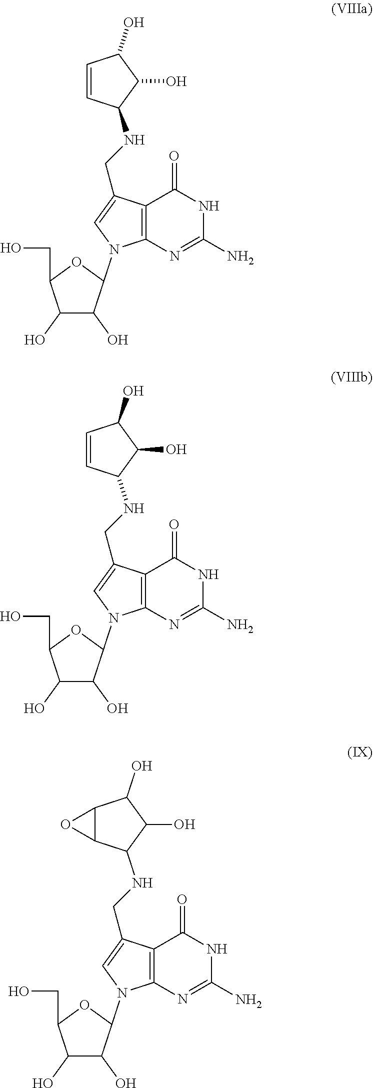 [SMILES: Nc1nc2c(c(CN[C@H]3C=C[C@H](O)[C@@H]3O)cn2C2OC(CO)C(O)C2O)c(=O)[nH]1 ; Nc1nc2c(c(CN[C@@H]3C=C[C@@H](O)[C@H]3O)cn2C2OC(CO)C(O)C2O)c(=O)[nH]1 ; Nc1nc2c(c(CNC3C(O)C(O)C4OC34)cn2C2OC(CO)C(O)C2O)c(=O)[nH]1]

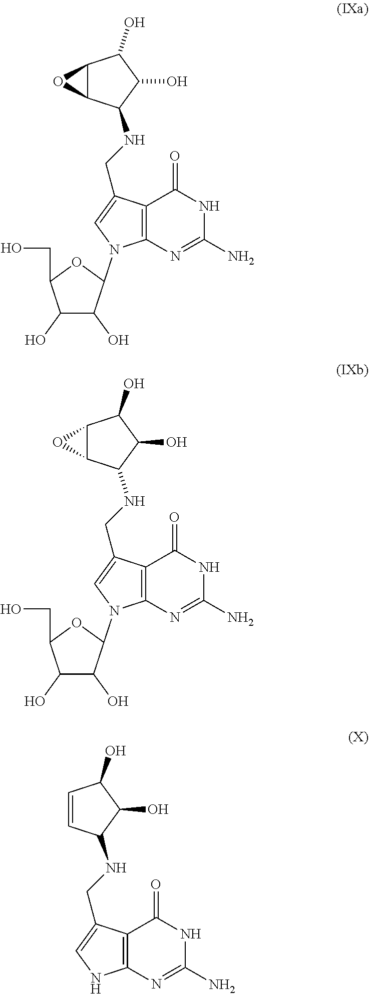

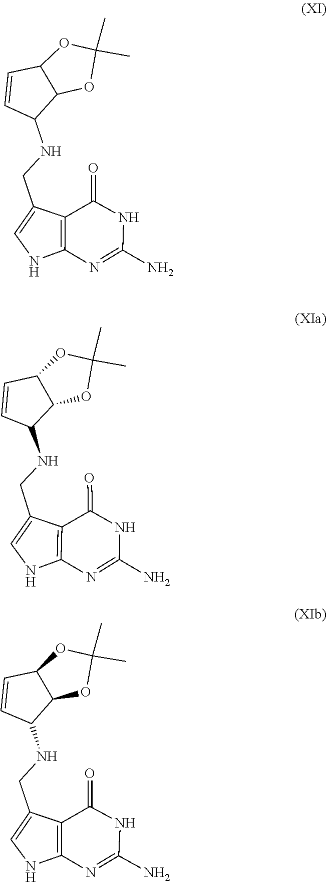

Preferably also, the compound of formula (I) according to the invention is represented by the following formulae (XII), (XIIa), (XIIb), (XIII), (XIV), (XV), (XVI), (XVII) or (XVIII):

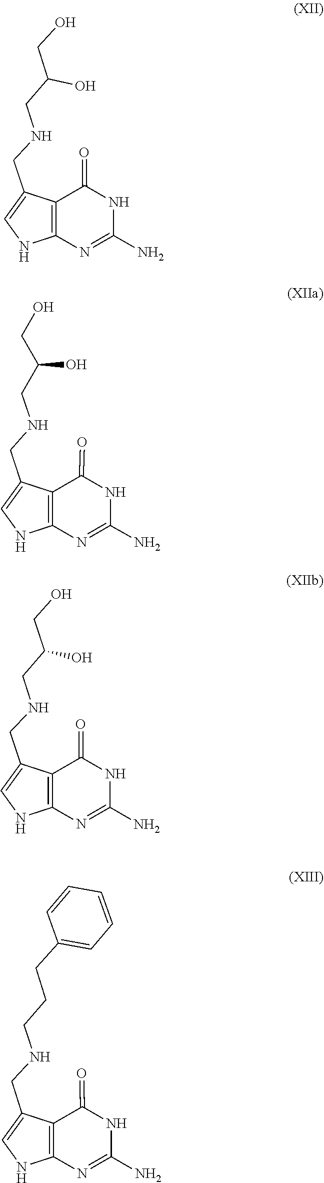 [SMILES: Nc1nc2[nH]cc(CNCC(O)CO)c2c(=O)[nH]1 ; Nc1nc2[nH]cc(CNC[C@H](O)CO)c2c(=O)[nH]1 ; Nc1nc2[nH]cc(CNC[C@@H](O)CO)c2c(=O)[nH]1 ; Nc1nc2[nH]cc(CNCCCc3ccccc3)c2c(=O)[nH]1]

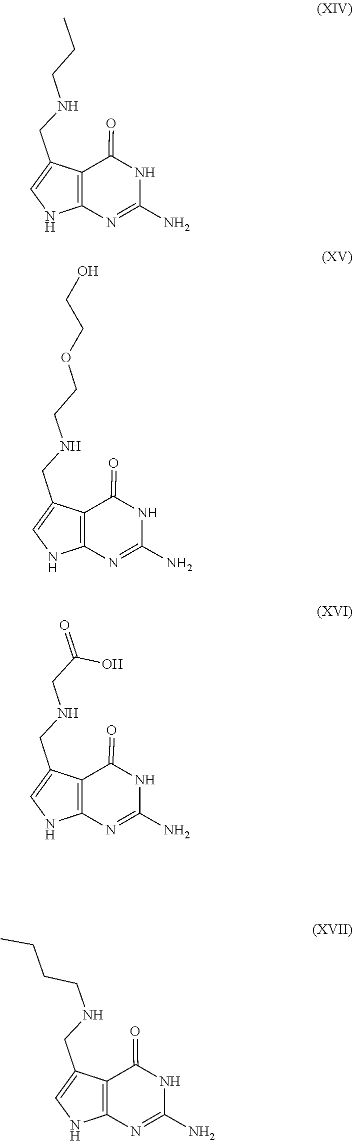 [SMILES: CCCNCc1c[nH]c2nc(N)[nH]c(=O)c12 ; Nc1nc2[nH]cc(CNCCOCCO)c2c(=O)[nH]1 ; Nc1nc2[nH]cc(CNCC(=O)O)c2c(=O)[nH]1 ; CCCCNCc1c[nH]c2nc(N)[nH]c(=O)c12]

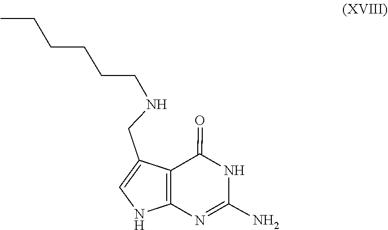 [SMILES: CCCCCCNCc1c[nH]c2nc(N)[nH]c(=O)c12]

The compound of formula (VIIa) is epoxyqueuine, also known as 7-(5-[3,4-epoxy-2,5-dihydroxycyclopent-1-yl)amino]methyl)-7-deazaguanine.

The compound of formula (Villa) is queuosine also known as 2-amino-5-({[(1S,4S,5R)-4,5-dihydroxycyclopent-2-en-1-yl]amino}methyl)-7-(3-D-ribofuranosyl)-1,7-dihydro-4H-pyrrolo[2,3-d]pyrimidin-4-one.

The compound of formula (IXa) is epoxyqueuosine also known as 7-(5-[(3,4-epoxy-2,5-dihydroxycyclopent-1-yl)amino]methyl)-7-deazaguanosine.

Preferably, the compound of formula (II) according to the invention is selected from the group consisting of mannosyl-queuine, galactosyl-queuine, glutamyl-queuine, galactosyl-queuosine, mannosyl-queuosine, glutamyl-queuosine, queuine-tRNA, and epoxyqueuine-tRNA.

The compound of formula (XIII) is N-((2-amino-4-oxo-4,7-dihydro-3Hpyrrolo[2,3-d]pyrimidin-5-yl)methyl)-3-phenylpropan-1-amine.

The compound (XIV) is N-((2-amino-4-oxo-4,7-dihydro-3Hpyrrolo[2,3-d]pyrimidin-5-yl)methyl)-propan-1-amine.

The compound (XVII) is N-((2-amino-4-oxo-4,7-dihydro-3Hpyrrolo[2,3-d]pyrimidin-5-yl)methyl)-butan-1-amine.

The compound (XVIII) is N-((2-amino-4-oxo-4,7-dihydro-3Hpyrrolo[2,3-d]pyrimidin-5-yl)methyl)-hexan-1-amine.

Preferably also the compound of formula (I), in particular the compound of formula (II), according to the invention is selected from the group consisting of queuine-tRNA^{Asp}, queuine-tRNA^{Tyr}, epoxyqueuine-tRNA^{Asp}, epoxyqueuine-tRNA^{Tyr}, queuine-tRNA^{Asn}, queuine-tRNA^{His}, epoxyqueuine-tRNA^{Asn}, epoxyqueuine-tRNA^{His}, mannosyl-queuine-tRNA^{Asp}, galactosyl-queuine-tRNA^{Tyr}, mannosyl-epoxyqueuine-tRNA^{Asp}, and galactosyl-epoxyqueuine-tRNA^{Tyr}.

Most preferably also the compound of formula (I), in particular the compound of formula (II), according to the invention (e.g., in the subject prophylactic methods, therapeutic methods, pharmaceutical compositions, products, and dietary supplements) is selected form the group consisting of queuine, ent-queuine, queuosine, epoxyqueuine, epoxyqueuosine, mannosyl-queuine, galactosyl-queuine, glutamyl-queuine, galactosyl-queuosine, mannosyl-queuosine, glutamyl-queuosine, queuine-tRNA and epoxyqueuine-Trna, a compound of formula (XII), (XIIa) and (XIIb).

### Neurodegenerative Disease

As used herein, “treating” a subject afflicted with a disorder shall include, without limitation, (i) slowing, stopping or reversing the progression of the disorder's symptoms, and/or (ii) reducing the likelihood that the disorder's symptoms will recur. In the preferred embodiment, treating a subject afflicted with a disorder means reversing the progression of the disorder's symptoms, ideally to the point of eliminating the symptoms.

As used herein, “preventing” a disorder in a subject includes, without limitation, reducing the likelihood of onset of the disorder's symptoms.

a-synucleinopathies according to the invention encompass neurodegenerative diseases characterised by the abnormal accumulation of a-synuclein aggregates in neurons, nerve fibres or glial cells. Preferably, the a-synucleinopathies are prevented or treated according to the invention by the inhibition of a-synuclein phosphorylation by the compound of formula (I), in particular the compound of formula (II), or the pharmaceutically acceptable salt or hydrate thereof according to the invention.

a-synucleinopathies notably encompass Lewy body disease and Multiple System Atrophy (MSA).

However, in an embodiment of the invention, the a-synucleiopathy prevented or treated according to the invention is not Multiple System Atrophy.

Besides, in another embodiment of the invention, the a-synucleiopathy prevented or treated according to the invention is not Parkinson's disease.

In yet another embodiment of the invention, the a-synucleiopathy prevented or treated according to the invention is not multiple sclerosis.

Preferably, the a-synucleiopathy prevented or treated according to the invention is Lewy body disease.

Lewy body disease is well known to one of skilled in the art. It is notably defined by class 8A22 of the 11^{th} revision of the International Classification of Diseases (ICD-11) set by the World Health Organization. In addition, Lewy body disease is defined by the following diagnostic criteria in the fifth edition of the Diagnostic and Statistical Manual of Mental Disorder (DSM-5) (2013) American Psychiatric Association, pages 618-621:
A. The criteria are met for major or mild neurocognitive disorder.
B. The disorder has an insidious onset and gradual progression.
C. The disorder meets a combination of core diagnostic features and suggestive diagnostic features for either probable or possible neurocognitive disorder with Lewy bodies.
   For probable major or mild neurocognitive disorder with Lewy bodies, the individual has two core features, or one suggestive feature with one or more core features.
   For possible major or mild neurocognitive disorder with Lewy bodies, the individual has only one core feature, or one or more suggestive features.
   1. Core diagnostic features:
      a. Fluctuating cognition with pronounced variations in attention and alertness.
      b. Recurrent visual hallucinations that are well formed and detailed.
      c. Spontaneous features of parkinsonism, with onset subsequent to the development of cognitive decline.
   2. Suggestive diagnostic features:
      a. Meets criteria for rapid eye movement sleep behavior disorder.
      b. Severe neuroleptic sensitivity.
D. The disturbance is not better explained by cerebrovascular disease, another neurodegenerative disease, the effects of a substance, or another mental, neurological, or systemic disorder.

Lewy body disease prevented or treated according to the invention may in particular be a major neurocognitive disorder with Lewy bodies with or without behavioral disturbance, a possible major neurocognitive disorder with Lewy bodies with or without behavioral disturbance, a probable major neurocognitive disorder with Lewy bodies with or without behavioral disturbance, a mild neurocognitive disorder with Lewy bodies with or without behavioral disturbance, and dementia with Lewy bodies

Preferably, the prevention or treatment of Lewy body disease according to the invention relates to the prevention or treatment of at least one, more preferably several, most preferably all, disorders due to or associated to Lewy body disease, in particular selected from the group consisting of cognitive or neurocognitive disorder due to Lewy bodies, such as dementia, visual hallucinations, symptoms of rapid eye movement (REM) sleep behavior disorder, hallucinations in other sensory modalities, depression, delusions, repeated falls and syncope and transient, episodes of unexplained loss of consciousness, autonomic dysfunction, such as orthostatic hypotension and urinary incontinence.

The invention also relates to the prevention or treatment of symptoms of Lewy body disease.

Huntington's disease is well known to one of skilled in the art. Huntington's disease prevented or treated according to the present invention notably encompasses Huntington's disease, dementia due to Huntington's disease and neurocognitive disorder due to Huntington's disease. Huntington's disease is notably defined by class 8A01.10 and dementia due to Huntington disease is notably defined by class 6D85.1 of the 11^{th} revision of the International Classification of Diseases (ICD-11) set by the World Health Organization. In addition, Huntington's disease is defined by the following diagnostic criteria in the fifth edition of the Diagnostic and Statistical Manual of Mental Disorder (DSM-5) (2013) American Psychiatric Association, pages 638-641:
A. The criteria are met for major or mild neurocognitive disorder.
B. There is insidious onset and gradual progression.
C. There is clinically established Huntington's disease, or risk for Huntington's disease based on family history or genetic testing.
D. The neurocognitive disorder is not attributable to another medical condition and is not better explained by another mental disorder.

Huntington's disease prevented or treated according to the invention may in particular be a major neurocognitive disorder due to Huntington's disease, with or without behavioural disturbance, and a mild neurocognitive disorder due to Huntington's disease, with or without behavioural disturbance.

Preferably, the prevention or treatment of Huntington's disease according to the invention relates to the prevention or treatment of at least one, more preferably several, most preferably all, disorders due or associated to Huntington's disease, in particular selected from the group consisting of cognitive or neurocognitive disorders such as behavioral changes and motor abnormalities.

By way of example, behavioural changes include irritability, anxiety, depressed mood pronounced apathy, disinhibition, impulsivity, and impaired insight.

By way of example, motor abnormalities include bradykinesia, dystonia, and rigidity.

The invention also relates to the prevention or treatment of symptoms of Huntington's disease.

Frontotemporal neurocognitive disorder is well known toy one of skilled in the art. Frontotemporal neurocognitive disorder according to the invention notably encompasses frontotemporal dementia. It is notably defined by classes 6D83 and 8A23 of the 11^{th} revision of the International Classification of Diseases (ICD-11) set by the World Health Organization. In addition, frontotemporal neurocognitive disorder is defined by the following diagnostic criteria in the fifth edition of the Diagnostic and Statistical Manual of Mental Disorder (DSM-5) (2013) American Psychiatric Association, pages 618-621:
A. The criteria are met for major or mild neurocognitive disorder.
B. The disturbance has insidious onset and gradual progression.
C. Either (1) or (2):
   1. Behavioral variant:
      a. Three or more of the following behavioral symptoms:
         i. Behavioral disinhibition.
         ii. Apathy or inertia.
         iii. Loss of sympathy or empathy.
         iv. Perseverative, stereotyped or compulsive/ritualistic behavior.
         v. Hyperorality and dietary changes.
      b. Prominent decline in social cognition and/or executive abilities.
   2. Language variant:
      a. PromiQent decline in language ability, in the form of speech production, word finding, object naming, grammar, or word comprehension.
D. Relative sparing of learning and memory and perceptual-motor function.
E. The disturbance is not better explained by cerebrovascular disease, another neurodegenerative disease, the effects of a substance, or another mental, neurological, or systemic disorder.
   Probable frontotemporal neurocognitive disorder is diagnosed if either of the following is present; otherwise, possible frontotemporal neurocognitive disorder should be diagnosed:
   1. Evidence of a causative frontotemporal neurocognitive disorder genetic mutation, from either family history or genetic testing.
   2. Evidence of disproportionate frontal and/or temporal lobe involvement from neuroimaging.
   Possible frontotemporal neurocognitive disorder is diagnosed if there is no evidence of a genetic mutation, and neuroimaging has not been performed.

Frontotemporal neurocognitive disorder prevented or treated according to the invention may in particular be a major frontotemporal neurocognitive disorder, a mild frontotemporal neurocognitive disorder, a probable major neurocognitive disorder due to frontotemporal lobar degeneration, a probable mild neurocognitive disorder due to frontotemporal lobar degeneration, a possible major neurocognitive disorder due to frontotemporal lobar degeneration, a mild neurocognitive disorder due to frontotemporal lobar degeneration.

Preferably, the prevention or treatment of frontotemporal neurocognitive disorder according to the invention relates to the prevention or treatment of at least one, more preferably several, most preferably all disorders due to or associated to frontotemporal neurocognitive disorder, in particular selected from the group consisting of cognitive or neurocognitive disorder due to frontotemporal neurocognitive disorder, such as behavioral and personality change, language impairment, behavioral variant apathy, disinhibition, lost of interest in socialization, selfcare, and personal responsibilities, socially inappropriate behaviors, changes in social style and in religious and political beliefs, hoarding, changes in eating behavior, hyperorality, loss of sphincter control, lack of planning and organization, distractibility, and poorjudgment.

The invention also relates to the prevention or treatment of symptoms of frontotemporal neurocognitive disorder.

Amyotrophic lateral sclerosis (ALS) is well known to one of skilled in the art. It is notably defined by class 8B60.0 of the 11^{th} revision of the International Classification of Diseases (ICD-11) set by the World Health Organization.

The Escorial criteria (Brooks et al. (2000) Amyotroph Lateral Scler Other Motor Neuron Disord. 1: 293-9) were developed to standardize diagnosis of ALS. These criteria include:
A. The presence of all of the following:
1. Evidence of lower motor neuron (LMN) degeneration by clinical, electrophysiologic, or neuropathologic examination.
2. Evidence of upper motor neuron (UMN) degeneration by clinical examination.
3. Progressive spread of symptoms or signs within a region or to other regions, as determined by history or examination.
B. Together with the absence of both of the following:
1. Electrophysiologic or pathologic evidence of other disease processes that could explain the signs of LMN and/or UMN degeneration.
2. Neuroimaging evidence of other disease processes that might explain the observed clinical and electrophysiologic signs.
   The clinical diagnosis of ALS, without pathologic confirmation, may be categorized into various levels of certainty by clinical and laboratory assessment based on the Escorial criteria (Brooks et al. (2000) Amyotroph Lateral Scler Other Motor Neuron Disord. 1: 293-9):
A. Clinically definite ALS: The presence of UMN and LMN signs in three regions.
B. Clinically definite familial, laboratory-supported ALS: Progressive upper and/or lower motor neuron signs in at least a single region (in the absence of another cause for the abnormal neurologic signs) in an individual with an identified ALS-causing variant.
C. Clinically probable ALS: The presence of UMN signs and LMN signs in at least two regions with some UMN signs necessarily rostral to (i.e., above) the LMN signs.
D. Clinically probable, laboratory-supported ALS: Clinical signs of UMN and LMN dysfunction are in only one region, or UMN signs alone present in one region, and LMN signs defined by electromyogram (EMG) criteria present in at least two limbs.
E. Clinically possible ALS: Clinical signs of UMN and LMN dysfunction found together in only one region, UMN signs found alone in two or more regions or LMN signs found rostral to UMN signs when the diagnosis of clinically probable, laboratory-supported ALS cannot be established.
F. Clinically suspected ALS: A pure LMN syndrome.

Amyotrophic lateral sclerosis prevented or treated according to the invention may in particular be a definite ALS, a probable ALS, a possible ALS and a suspected ALS.

Preferably, the prevention or treatment of amyotrophic lateral sclerosis according to the invention relates to the prevention or treatment of at least one, more preferably several, most preferably all, disorders due to or associated to ALS, in particular selected from the group consisting of cognitive or neurocognitive disorder due to amyotrophic lateral sclerosis, such as hyperreflexia, extensor plantar response, increased muscle tone, weakness in a topographic representation, weakness, muscle wasting, hyporeflexia, muscle cramps, and fasciculations.

The invention also relates to the prevention or treatment of symptoms of amyotrophic lateral sclerosis disorder.

### Individual

The individual according to the invention is preferably a human.

The individual according to the invention may present one or more symptoms of a neurodegenerative disease selected from the group consisting of a-synucleinopathy, in particular Lewy body disease, Huntington's disease, frontotemporal neurocognitive disorder, and amyotrophic lateral sclerosis.

### Additional Compound

The additional compound useful for the prevention or treatment of a neurodegenerative disease according to the invention can be of any type known of one of skilled in the art. Preferably, the additional compound according to the invention is selected from the group consisting of tetrabenazine, risperidone, olanzapine, citalopram, fluoxstine, L-Dopa, carbidopa, donepezil, galantamine, memantine, and rivastigmine, trazodone, riluzole, edaravone, bromocriptine, cabergoline, pramipexole, ropinirole, pirebidil, lisurdide, apomorphine, selegiline, and rasagiline.

Preferably, the additional compound in the dietary supplement is selected from the group consisting of vitamins, minerals, fatty acids, amino acids, antioxidants and derivatives or precursors thereof.

Preferably vitamins are selected from the group consisting of pyridoxine, pyridoxal phosphate (Vitamin B₆), riboflavin, thiamine, vitamin E, vitamin K3, vitamin C, niacin, CoQ10 and β-carotene.

Preferably, minerals are selected from the group consisting of calcium, magnesium, selenium and phosphorus.

Preferably, the amino-acid is L-DOPA (levodopa).

Preferably, the fatty acids are selected from the group consisting of Levo-carnitine and acetyl-L-carnitine.

### Administration

As intended herein, “combined” or “in combination” means that the compound of formula (I), in particular the compound of formula (II) as defined above, is administered at the same time than another compound or product, either together, i.e. at the same administration site, or separately, or at different times, provided that the time period during which the compound of formula (I) as defined above exerts its effects on the individual and the time period during which the additional agent or product exerts its pharmacological effects on the individual, at least partially intersect.

Preferably also, the compound of formula (I), in particular the compound of formula (II), according to the invention or the pharmaceutically acceptable salt or hydrate thereof is for an administration or is administered at a dosage regimen of from 0.01 to 40 mg/kg/d, more preferably of from 0.01 to 10 mg/kg/d, even more preferably of from 0.01 to 1 mg/kg/d, and most preferably of from 0.01 to 0.1 mg/kg/d. In an additional embodiment, the compound of formula (I), in particular the compound of formula (II), and most preferably queuine, according to the invention or the pharmaceutically acceptable salt or hydrate thereof is for an administration or is administered at a dosage regimen of (i) 0.01 mg/kg/d, 0.02 mg/kg/d, 0.03 mg/kg/d, 0.04 mg/kg/d, 0.05 mg/kg/d, 0.06 mg/kg/d, 0.07 mg/kg/d, 0.08 mg/kg/d, 0.09 mg/kg/d, or 0.1 mg/kg/d; or (ii) from 0.01 to 0.02 mg/kg/d, from 0.02 to 0.03 mg/kg/d, from 0.03 to 0.04 mg/kg/d, from 0.04 to 0.05 mg/kg/d, from 0.05 to 0.06 mg/kg/d, from 0.06 to 0.07 mg/kg/d, from 0.07 to 0.08 mg/kg/d, from 0.08 to 0.09 mg/kg/d, or from 0.09 to 0.1 mg/kg/d. For the subject therapeutic methods, the amounts exemplified in this paragraph are “therapeutically effective amounts.” For the subject prophylactic methods, the amounts exemplified in this paragraph are “prophylactically effective amounts.”

Preferably, the compound of formula (I), in particular the compound of formula (II) according to the invention or the pharmaceutically acceptable salt or hydrate thereof is in a form suitable for an administration or is administered by the oral route, the intradermal route, the intravenous route, the intramuscular route or the subcutaneous route. Preferably, the compound of formula (I), in particular the compound of formula (II), according to the invention, or the pharmaceutical composition, medicament, products or dietary supplement comprising it is in a form suitable for an administration or is administered by a hypodermic implant.

Preferably, the compound of formula (I) according to the invention, or the pharmaceutical composition, medicament, products or dietary supplement comprising it is in the form of a powder, sachets, tablets, gelatine, capsules, or a liquid or gel solution.

Preferably also, the pharmaceutical composition, medicament, products or dietary supplement according to the invention, comprises the compound of formula (I) according to the invention, in particular queuine, ent-queuine, queuosine, the compound of formula (XII), (XIIa), or (XIIb) at a unit dose of at least 0.15 mg, at least 1 mg, at least 10 mg, at least 50 mg, at least 100 mg, at least 500 mg or at least 1000 mg.

Preferably also, the pharmaceutical composition, medicament, products or dietary supplement according to the invention, comprises an extract, in particular a purified extract, from microorganism and/or plant, which comprises the compound of formula (I) according to the invention, in particular queuine, ent-queuine, queuosine, the compound of formula (XII), (XIIa), or (XIIb) in particular at a unit dose of (i) at least 0.15 mg, at least 1 mg, at least 10 mg, at least 50 mg, at least 100 mg, at least 200 mg, at least 500 mg or at least 1000 mg, or (ii) from 0.2 mg to 0.5 mg, from 0.5 mg to 1 mg, from 1 mg to 10 mg, from 10 mg to 50 mg, from 50 mg to 100 mg, from 100 mg to 200 mg, from 200 mg to 500 mg, or from 500 mg to 1000 mg.

### Additional Embodiments

The present invention further provides a method for causing a neuroprotective effect (e.g., protecting against alpha-synuclein aggregation) in an individual having an alpha-synucleinopathy (e.g., Lewy body disease, Huntington's disease, frontotemporal neurocognitive disorder, or amyotrophic lateral sclerosis) comprising administering to the individual queuine or a pharmaceutically acceptable salt or hydrate thereof. In one embodiment, the queuine or pharmaceutically acceptable salt or hydrate thereof is administered in a dosage regimen of from 0.01 mg/kg/d to 40 mg/kg/d; from 0.01 mg/kg/d to 10 mg/kg/d; from 0.01 mg/kg/d to 1 mg/kg/d; or from 0.01 mg/kg/d to 0.1 mg/kg/d. In another embodiment, the queuine or pharmaceutically acceptable salt or hydrate thereof is administered orally, intradermally, intravenously, intramuscularly, or subcutaneously.

In a further embodiment, the queuine or pharmaceutically acceptable salt or hydrate thereof is administered in combination with a compound selected from the group consisting of tetrabenazine, risperidone, olanzapine, citalopram, fluoxetine, L-Dopa, carbidopa, donepezil, galantamine, memantine, and rivastigmine, trazodone, riluzole, edaravone, bromocriptine, cabergoline, pramipexole, ropinirole, pirebidil, lisurdide, apomorphine, selegiline, and rasagiline.

In a further embodiment, the queuine or pharmaceutically acceptable salt or hydrate thereof is administered in the form of a pharmaceutical composition comprising at least one pharmaceutically acceptable excipient or vehicle and queuine. The pharmaceutical composition optionally comprises a compound selected from the group consisting of tetrabenazine, risperidone, olanzapine, citalopram, fluoxetine, L-Dopa, carbidopa, donepezil, galantamine, memantine, and rivastigmine, trazodone, riluzole, edaravone, bromocriptine, cabergoline, pramipexole, ropinirole, pirebidil, lisurdide, apomorphine, selegiline, and rasagiline.

In a further embodiment, the queuine or pharmaceutically acceptable salt or hydrate thereof is formulated as a dietary supplement. This dietary supplement optionally comprises additional compounds selected from the group consisting of vitamins, minerals, fatty acids, amino acids, and antioxidants.

The present invention will be further explained by the following non-limiting examples.

### DESCRIPTION OF THE FIGURES

FIG. 1

FIG. 1 represents an immunofluorescence microscope photograph of WT mouse cortical neurons exposed to 10 nM of huPFFs, stained with the EP1536Y antibody which specifically detects the S129 phosphorylated form of a-synuclein, and either pre-treated with 100 nM queuine (right panel) or not treated (control, left panel).

FIG. 2

FIG. 2 represents the quantification of the EP1536Y signal and Lewy neurite length after normalisation with the huPFF condition (huPFFs, no compound) in WT cortical neurones (vertical axis) as a function of queuine concentration (horizontal axis). The data are expressed as mean of group and SEM. Data were analysed using two-way ANOVA followed by Dunnett's multiple comparison; ***p<0.001 cf. huPFFs+0 μM group

### EXAMPLE

The study was designed to evaluate the effects of queuine in an in vitro cell-based experimental model of synucleinopathy. The study aimed to evaluate the impact of queuine on the experimental synucleinopathy, in particular of the Lewy body type, induced by exogenous human preformed a-synuclein fibrils (huPFFs) in cortical cultures of wild-type (WT) mice (Volpicelli-Daley et al. (2011) Neuron. 72:57-7).

Material and Methods

Queuine was evaluated for its capability to afford protection against the induction of endogenous a-synuclein phosphorylation at serine 129 (a surrogate marker of de novo a-synuclein aggregation) triggered by exposure to exogenous human recombinant (unphosphorylated) a-synuclein preformed fibrils (huPFFs).

Primary cultures of dissociated cortices from embryonic day E18 WT mouse embryos were used as the biological system. Timed pregnant female mice were received from Charles River Laboratories 2 days before initiation of the primary culture. Cortices were harvested from the E18 embryos of WT mice and dissociated enzymatically and mechanically to yield a homogenous cell suspension. 20000 cells were plated per well in poly-D-Lysine-coated 96-well plates in a neuronal medium containing 0.5% Penicillin/Streptomycin and 0.5 mM L-glutamine. The cultures were incubated at 37° C./5% CO2. At DIV 3 they were exposed to queuine applied at 3 concentrations (10 μM, 1 μM, 0.1 μM) or zero. Queuine treatment was then renewed every 3 days (by replacing ⅓ of the volume of medium with fresh medium supplemented with 1× concentration of QUEUINE). At DIV 7, half of the culture wells were additionally exposed to huPFFs (at 1 concentration, i.e. equivalent to 10 nM a-synuclein monomer, final concentration). At 30 DIV, the neuronal cultures were fixed with PFA, and the effects of the treatments were evaluated by performing two double immunostainings:
(i) D37A6/Syn1 to detect total rodent a-synuclein/human plus rodent non aggregated a-synuclein;
(ii) EP1536Y/Syn211 to detect rodent and human a-synuclein phosphorylated at S129/total human a-synuclein.

The induction of a-synuclein phosphorylation was quantified by measuring the accumulation of phospho-a-synuclein (length of the phospho-a-synuclein-positive neurite network, number of phospho-a-synuclein positive cell bodies).

The experimental conditions were performed in quadruplicate (for compound-treated conditions+/−huPFFs) and triplicate (for controls+/−huPFFs) wells. An array of nine individual microscopic fields were acquired in each single well, and 3 channels were recorded for each field (green fluorescence excited @488 nm, red fluorescence excited @594 nm, and phase contrast).

Results

Endogenous a-Synuclein Detection

Immunofluorescence performed using D37A6 and syn-1 antibodies on WT mouse cortical cultures revealed the distribution and the amounts of endogenous murine a-synuclein. D37A6 specifically recognises the rodent form of the protein, principally expressed by neurones and distributed at the presynaptic sites, while it is less prone to hybridise with the phosphorylated/aggregated forms of the protein. The syn-1 antibody does not have a species specificity, but it is conformation sensitive: in non-denaturing conditions, it binds neither to the fibrillar (amyloid) form of the endogenous protein nor to the exogenously added huPFFs in which the epitope is hidden by its engagement into the amyloid structure.

In the case of the WT mouse primary neuronal cultures, both antibodies stain endogenous a-synuclein and their signals largely overlap.

The induction of a-synuclein de novo aggregation/phosphorylation upon exposure to huPFFs is thus expected to cause a decrease of the signals obtained by both the antibodies due to the recruitment of the detectable a-synuclein forms (non-amyloid ones) into less- or nondetectable aggregated assemblies (amyloid).

huPFFs Detection and Induced a-Synuclein Phosphorylation

Immunofluorescence performed using Syn-211 specifically allows the detection of human a-synuclein, even when engaged into amyloid structures (the only “invisible” forms are the Cter truncated ones). Thus, in the present experimental model this antibody could only bind the exogenous huPFFs, provided that human a-synuclein was not cleaved at its C-terminal. After three weeks of exposure, huPFFs were largely processed in the neurones and the syn211 signal tended to disappear. Human synuclein could, however, still be detected, associated with neurones, but also particularly with a non-neuronal subpopulation of cells (astrocytes) that accumulate huPFFs without processing them efficiently.

The EP1536Y antibody specifically detects the S129 phosphorylated form of a-synuclein (both human and rodent). In WT primary cultures of cortical neurones, a-synuclein phosphorylation is (i) specifically induced by PFFs exposure, (ii) associated with a-synuclein aggregation and (iii) is restricted to the endogenous protein (the huPFFs do not get phosphorylated at S129). EP1536Y staining shows the appearance of Lewy neurites and a-synuclein aggregates in the neuronal cell bodies, all of them caused by huPFFs exposure. No signal is observed in the controls.

Effects of Queuine on huPFFs Induced a-Synuclein Phosphorylation

Exposure of the neurones to 10 nM huPFFs for 21 or 23 days induced the phosphorylation of endogenous a-synuclein.

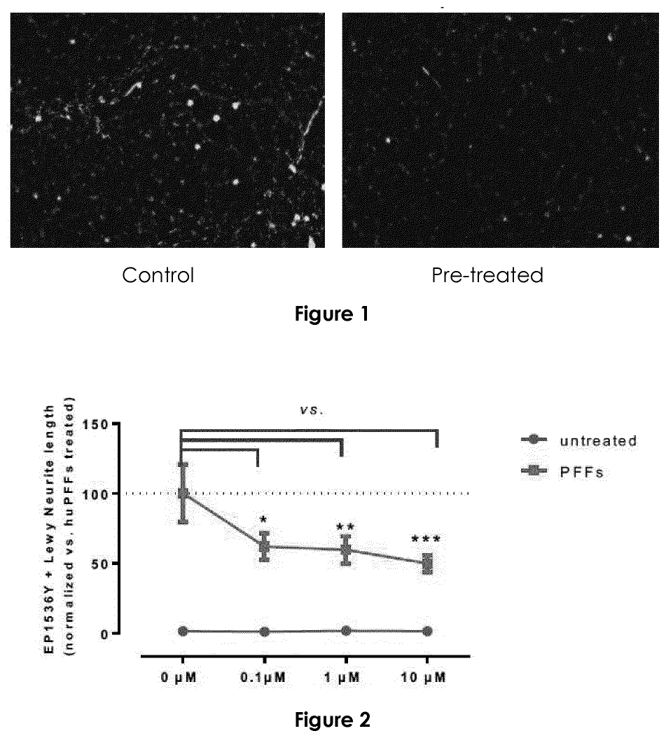

The EP1536Y immunofluorescence signal (in green), representative of the phosphorylation of a-synuclein, is decreased by queuine pretreatment at 100 nM compared to control (FIG. 1), thereby showing that queuine inhibits the phosphorylation of a-synuclein induced by huPFFs.

To further evaluate the effects of queuine, the EP1536Y signal was segmented to quantify the phospho-a-synuclein positive neurites. The results were normalised with regards to the huPFFs-treated condition to appreciate the effects of the compound on the PFFs-induced phosphorylation (FIG. 2).

In the concentration range tested, queuine had no effect on the untreated neurones, i.e. it did not induce any synuclein phosphorylation at S129. In huPFFs-treated neurones, and at all doses tested, queuine antagonised the effects of PFFs, and induced a significant decrease of a-synuclein phosphorylation at S129 (approx. −50%) induced by huPFFs.

1.-16. (canceled)

17. A method for preventing or treating a neurodegenerative disease in an individual comprising administering to the individual an effective amount of a compound of formula (I) or a pharmaceutically acceptable salt or hydrate thereof, wherein the neurodegenerative disease is selected from the group consisting of a-synucleinopathy, in particular Lewy body disease, Huntington's disease, frontotemporal neurocognitive disorder, and amyotrophic lateral sclerosis, and wherein the compound of formula (I) has the following structure: 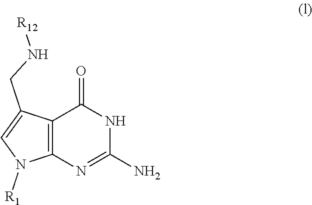
wherein:
R₁ represents —H or a ribosyl group of the following formula: 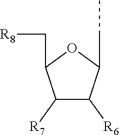
wherein:
R₆ represents —H; —O—R₉ or —O—CO—R₉ wherein R₉ is H, an alkyl group having from 1 to 6 carbon atoms or an aryl group having from 3 to 12 carbon atoms;
R₇ represents —H; —O—R₁₀ or —O—CO—R₁₀ wherein R₁₀ is H, an alkyl group having from 1 to 6 carbon atoms or an aryl group having from 3 to 12 carbon atoms; a deoxyribonucleic acid group; or a ribonucleic acid group;
R₈ represents —H; —O—R₁₁ or —O—CO—R wherein R₁₁ is H, an alkyl group having from 1 to 20 carbon atoms or an aryl group having from 3 to 20 carbon atoms; a phosphate group; a diphosphate group; a triphosphate group; a deoxyribonucleic acid group; or a ribonucleic acid group; and
R₁₂ represents a saturated or unsaturated alkyl, cycloalkyl, heterocycloalkyl or ether group having from 1 to 20 carbon atoms, optionally substituted by at least one group selected from the group consisting of:
an alkyl group having from 1 to 20 carbon atoms,
an aryl or heteroaryl group having from 3 to 20 carbon atoms,
a cycloalkyl or heterocycloalkyl group having from 3 to 20 carbon atoms,
a hydroxyl group,
a carbonyl or carboxyl group having from 1 to 20 carbon atoms,
an epoxy group,
an —O—R₄ group wherein R₄ is H, an alkyl group having from 1 to 6 carbon atoms, an aryl group having from 3 to 12 carbon atoms, a glycosyl group or an aminoacyl group, and
an —O—CO—R₅ group wherein R₅ is an alkyl group having from 1 to 6 carbon atoms, an aryl group having from 3 to 12 carbon atoms or a glycosyl group.

18. The method of claim 17, wherein the compound of formula (I) is of the following formula (II): 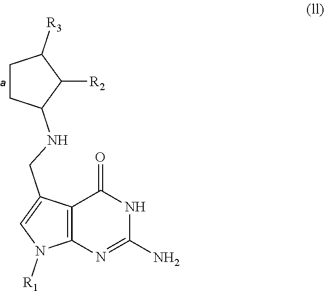
wherein:
a represents a double bond or an epoxy group, and
R₁ represents —H or a ribosyl group of the following formula: 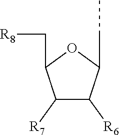
wherein:
R₆ represents —H; —O—R₉ or —O—CO—R₉ wherein R₉ is H, an alkyl group having from 1 to 6 carbon atoms or an aryl group having from 3 to 12 carbon atoms;
R₇ represents —H; —O—R₁ₒ or —O—CO—R₁₀ wherein R₁₀ is H, an alkyl group having from 1 to 6 carbon atoms or an aryl group having from 3 to 12 carbon atoms; a deoxyribonucleic acid group; or a ribonucleic acid group;
R₈ represents —H; —O—R₁₁ or —O—CO—R₁₁ wherein R₁₁ is H, an alkyl group having from 1 to 20 carbon atoms or an aryl group having from 3 to 20 carbon atoms; a phosphate group; a diphosphate group; a triphosphate group; a deoxyribonucleic acid group; or a ribonucleic acid group; and
R₂ and R₃, which are identical or different, represent —O—R₄ wherein R₄ is H, an alkyl group having from 1 to 6 carbon atoms, an aryl group having from 3 to 12 carbon atoms, a glycosyl group or an aminoacyl group; or —O—CO—R₅ wherein R₅ is an alkyl group having from 1 to 6 carbon atoms, an aryl group having from 3 to 12 carbon atoms or a glycosyl group.

19. The method of claim 18, wherein:
R₂ and R₃, which are identical or different, represent —OH, a —O-mannosyl group, a —O-galactosyl group or a —O-glutamyl group;
R₆ represents —OH; and
R₇ and R₈, which are identical or different, represent —OH or a ribonucleic acid group.

20. The method of claim 17, wherein the compound is selected form the group consisting of queuine, ent-queuine, queuosine, epoxyqueuine, epoxyqueuosine, mannosyl-queuine, galactosyl-queuine, glutamyl-queuine, galactosyl-queuosine, mannosyl-queuosine, glutamyl-queuosine, queuine-tRNA and epoxyqueuine-tRNA.

21. The method of claim 17, wherein the compound is selected from the group consisting of the compounds of the following formulae:

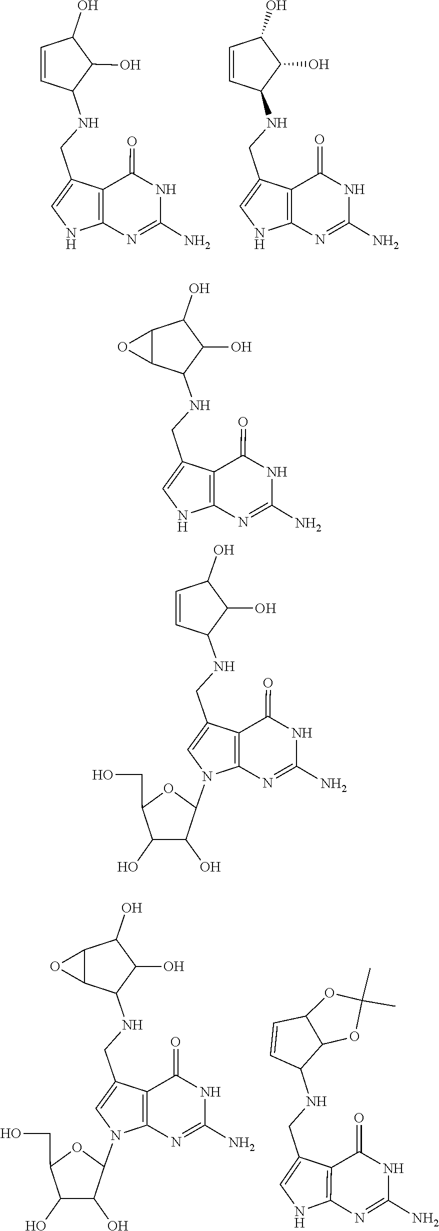

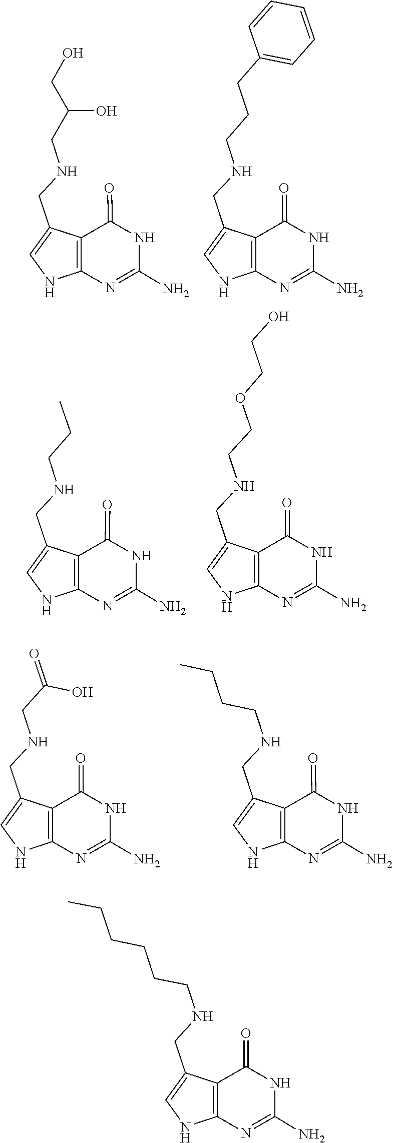

22. The method of claim 17, wherein the compound or pharmaceutically acceptable salt or hydrate thereof is administered at a unit dose of from 5 to 1,500 mg/kg.

23. The method of claim 17, wherein the compound or pharmaceutically acceptable salt or hydrate thereof is administered at a dosage regimen of from 0.01 to 40 mg/kg/d.

24. The method of claim 17, wherein the compound or pharmaceutically acceptable salt or hydrate thereof is formulated for administration by the oral route, the intradermal route, the intravenous route, the intramuscular route or the subcutaneous route.

25. The method of claim 17, wherein the compound or pharmaceutically acceptable salt or hydrate thereof is administered in combination with at least one additional compound useful for the prevention or treatment of a neurodegenerative disease selected from the group consisting of a-synucleinopathy, in particular Lewy body disease, Huntington's disease, frontotemporal neurocognitive disorder, and amyotrophic lateral sclerosis.

26. The method of claim 25, wherein the at least one additional compound is selected from the group consisting of tetrabenazine, risperidone, olanzapine, citalopram, fluoxetine, L-Dopa, donepezil, galantamine, memantine, rivastigmine, trazodone, riluzole, edaravone, bromocriptine, cabergoline, pramipexole, ropinirole, pirebidil, lisurdide, apomorphine, selegiline, and rasagiline.

27. The method of claim 25, wherein the compound or pharmaceutically acceptable salt or hydrate thereof is administered as a combined preparation with the at least one additional compound.

28. The method of claim 25, wherein the compound or pharmaceutically acceptable salt or hydrate thereof is administered in a separate manner with respect to the at least one additional compound.

29. The method of claim 17, wherein the compound or pharmaceutically acceptable salt or hydrate thereof is administered in association with at least one pharmaceutically acceptable excipient or vehicle.

30. The method of claim 17, wherein the compound or pharmaceutically acceptable salt or hydrate thereof is administered in the form of a dietary supplement.

31. The method of claim 30, wherein the dietary supplement further comprises additional compounds selected from the group consisting of vitamins, minerals, fatty acids, amino acids and antioxidants.
